(19) United States
(12) Plant Patent Application Publication
(10) Pub. No.: US 2002/0010954 P1
(43) Pub. Date: Jan. 24, 2002

(54) Chrysanthemum named 'Cream Elite Reagan'

(76) Inventor: Robert Noodelijk, Woubrugge (NL)

Correspondence Address: PARKHURST & WENDEL, SUITE 210, 1421 PRINCE STREET, ALEXANDRIA, VA, 22314, US

(21) Appl. No.: 09/902,750

(22) Filed: Jul. 12, 2001

(30) Foreign Application Priority Data
Apr. 19, 1999 (NL) CHR 3118
Jul. 28, 2000 (EP) 2000/11172

(51) Int. Cl. A01H 5/00

(52) U.S. Cl. PLT/294

(57) ABSTRACT

A Chrysanthemum plant named ‘Cream Elite Reagan’ characterized by its medium sized blooms with pale yellow ray-florets and yellow-green disc florets.

### RELATED CULTIVARS

‘Cream Elite Reagan’ is related to ‘White Elite Reagan’ (#______) and the co-pending application of ‘Sunny Elite Reagan’. ‘Cream Elite Reagan’ and ‘Sunny Elite reagan’ are both natural mutants of ‘White Elite Reagan’,

### BACKGROUND OF THE INVENTION

‘Cream Elite Reagan’ is a product of a breeding and selection program which had the objective of fmding color mutants of ‘White Elite Reagan’. The new plant of the present invention comprises a new and distinct cultivar of chrysanthemum plant that is a natural occuring sport of a parent chrysanthemum named ‘White Elite Reagan’ ( #______). A comparison with Parent chrysanthemum ‘White Elite Reagan’ is also made in this application. The new cultivar was discovered as a whole plant mutation in March 1999 by Rob Noodelijk in a controlled environment (greenhouse) in Rijsenhout Holland. The first act of asexual reproduction of ‘Cream Elite Reagan’ was accomplished when vegetative cuttings were taken from the initial selection in May 1999 in Rijsenhout Holland.

### SUMMARY OF THE INVENTION

The present invention is a new and distinct variety of chrysanthemum bearing medium sized blooms with pale yellow ray-florets and yellow-green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

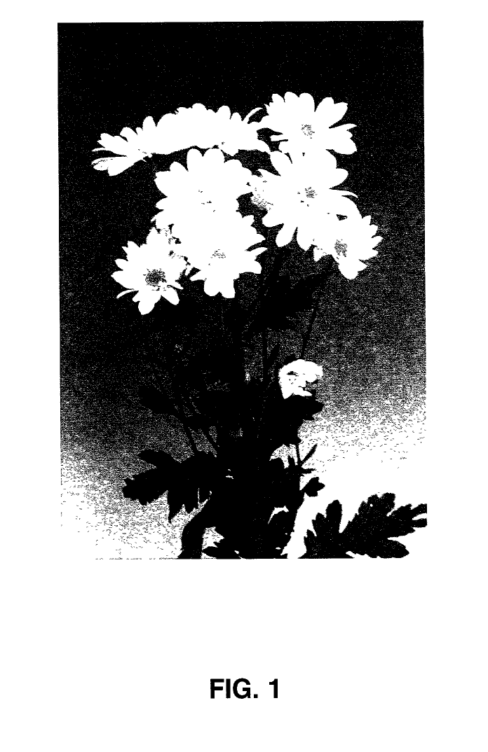

FIG. 2 shows the various stages of bloom of the new cultivar.

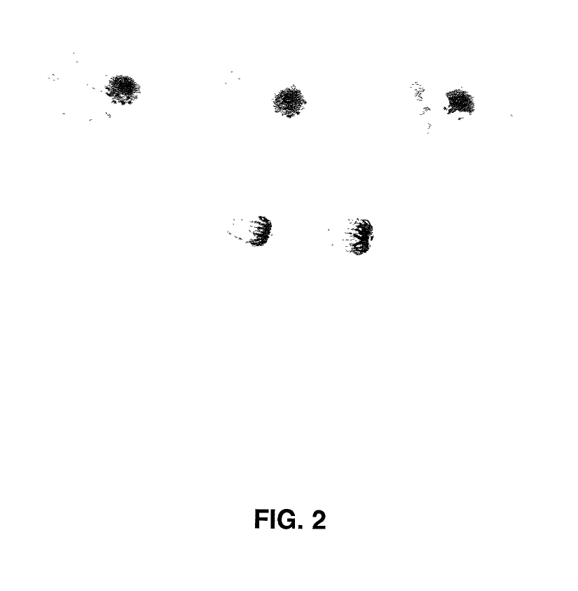

FIG. 3 shows the foliage of the new cultivar.

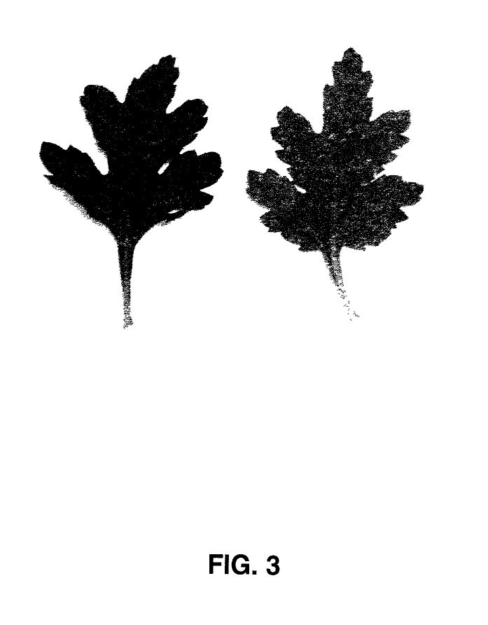

### DESCRIPTION OF THE INVENTION

This new variety of chrysanthemum is of the botanical classification dendranthema grandiflora. The observations and measurements were gathered from plants grown in April/May in a greenhouse in Rijsenhout Holland in a photo-periodic controlled crop under conditions generally used in commercial practice. The greenhouse temperatures during this crop were at day-time between 18° C. and 25° C. and at night 20° C. After a long day period of 14 days the photo-periodic response time in this crop was 48 days. After the long day period to flowering growth retardants were applied 2 to 3 times in an average dose of 2.5 gram/liter water. No tests were done on disease or insect resistance or susceptibility. This new variety produces medium sized blooms with pale yellow ray-florets and yellow-green disc-florets blooming on the plant for 5 weeks. This new variety of chrysanthemum has been found to retain its distinctive characteristics throughout successive propagations however the phenotype may vary significantly with variations in environment such as light intensity and temperature. To show the phenotype as described ‘Cream Elite Reagan’ can be planted without assimilation lightning (high pressure sodium lamps) between week 50 and week 40 of the next year under greenhouse conditions in Holland. With assimilation light (minimum level 2500 lux) it can be planted year round under greenhouse conditions in Holland. From the cultivars known to inventor the most similar existing cultivar in comparison to ‘Cream Elite Reagan’ is ‘White Elite Reagan’ and ‘Cream Reagan’. When ‘White Elite Reagan’ and ‘Cream Elite Reagan’ are being compared the following differences and similarities arenoticed: The difference of ‘White Elite Reagan’ and ‘Cream Elite Reagan’ is the flower color for which characteristic ‘Cream Elite Reagan’ has been selected out of ‘White Elite Reagan’. All other characteristics of ‘White Elite Reagan’ and ‘Cream Elite Reagan’ are similar. The differences of ‘Cream Elite Reagan’ and ‘Cream Reagan’ are: (1) Disc flower color and size. The flower disc of ‘Cream Elite Reagan’ is more green and smaller.(2) Growth rate. The growth of ‘Cream Elite Reagan’ is more vigorous. (3) Response time. The response time of ‘Cream Elite Reagan’ is 3 till 4 days shorter.

The following is a description of the plant and characteristics that distinguish ‘Cream Elite Reagan’ as a new and distinct variety.

The color designations are taken from the plant itself. Accordingly, any discrepancies between the color designations and the colors depicted in the photographs are due to photographic tolerances. The color chart used in this description is: The Royal Horticultural Society color chart, edition 1995.

TABLE 1
Botanical Description of
CULTIVAR ‘Cream Elite Reagan’

| | |
|---|---|
| Bud | |
| Size | Medium, cross-section +/− 1.0 cm |
| | height +/− 0.8 cm |
| Outside Color | Yellow 8 B |
| Involucral bracts | 2 rows, length 7 mm, width 3 mm |
| Involucral bracts among | Not present |
| disc-florets | |
| Involucral bracts color | Green 138 B |
| Bloom | |
| Type | Single |
| Height | Flat |
| Size | Medium to large |
| Fully Expanded | 7.5-8.0 cm |
| Number of blooms per single stem | Average of 10-12 |
| Seeds | Not produced |
| Performance (blooming period) | 26-28 days |
| Fragrance | Typical chrysanthemum |
| Color | |
| Center of the flower (disc-florets) | Immature yellow-green 144 B, |
| | mature yellow-green 144 B at the |
| | center to yellow-green 154 A |
| | along the border |
| Color of upper surface of the majo- | Yellow 5 D |
| rity of the ray-florets | |
| Color of the lower surface of the | Yellow 2C |
| majority of the ray-florets | |
| Tonality from Distance | A spray mum with pale yellow |
| | flowers and a yellow-green disc |
| Discoloration to color | Yellow 4 D |
| Ray florets | |
| Texture | Upper and under side smooth |
| Number | 22-24 |
| Cross-section | Flat, sometimes convex |
| Longitudinal axis of majority | Straight, sometimes reflexing |
| Length of corolla tube | Very short to short |
| Ray-floret length | 3.5 cm |
| Ray-floret width | 1.2 cm |
| Ratio length/width | Low to medium |
| Shape of tip | Round sometimes dentate |
| Disc florets | |
| Disc diameter | Medium, 1.0-1.2 cm |
| Distribution of disc florets | Numerous, clearly visible at all stages |
| | of flowering |
| Type | Tubular |
| Color | Yellow-green 144 A |
| Receptacle shape | Conical raised |
| Reproductive Organs | |
| Stamen (present in disc florets | Yellow-green, thin 3 mm in length |
| only) | |
| Stamen color | Yellow-green 150 B |
| Pollen | Appears at a very late stage of |
| | flowering, |
| Pollen color | Close to yellow 12 A |
| Styles (present in both ray and disc | Green, thin |
| florets) | |
| Style color | Green 143 B |
| Style Length | 4 mm |
| Stigmas | Yellow-green |
| Stigma Width | 1 mm |
| Ovaries | Enclosed in calyx |
| Plant | |
| Form | A spray mum meant for erect culture |
| Growth habit | Upright |
| Growth rate | Vigorous |
| Height | 100-125 cm |
| internodes | 3.0 cm |
| Spray formation | Corymbiform (funnel-shaped with a |
| | little round to flat umbel on top) |
| Peduncle length | Near the top 14 cm, near the middle |
| | 23 cm, near the bottom 30 cm |
| Peduncle color | Green 138 B |
| Peduncle, attachment | Good, not brittle |
| Peduncle, angle with stem | Small, 30° |
| Stem Color | Green 138 B |
| Stem Strength | Medium |
| Stem Brittleness | Absent |
| Stem Anthocyanin Coloration | Absent |
| Flowering Response | 48 days |
| (photo-periodic controlled crop, | |
| not natural season) | |
| Foliage | |
| Color | Upper side Under side |
| Size | Medium; length +/− 12 cm, width |
| | +/− 8.5-9.0 cm. |
| Quantity (number per single stem) | 22-28 |
| Shape | Ovate to oval |
| Texture upper side | Scabrous |
| Texture under side | Slightly pubescent |
| Venation arrangement | Pinnate |
| Shape of the margin | Lobed |
| Shape of Base of Sinus Between | Round |
| Lateral Lobes | |
| Margin of Sinus Between Lateral | Parallel to diverging |
| Lobes | |
| Shape of Base | Truncate |
| Apex | Mucronate |

### TABLE 2

### Differences with the Comparison Varieties

TABLE 2
Differences with the comparison Varieties

| | ‘Cream | ‘White | |
|---|---|---|---|
| | Elite Reagan’ | Elite Reagan’ | ‘Cream Reagan’ |
| Color of the | Yellow 5 D | White 155 D | Yellow 5 A |
| ray-florets | | | |
| Flower disc size | 1.0-1.2 cm | 1.0-1.2 cm | 1.5-2.0 cm |
| Flower disc color | Yellow-green | Yellow-green | Yellow-green |
| immature | 144 B | 144 A | 151 C |
| Growth rate | Vigorous | Vigorous | Medium |
| Peduncle, angle | 30° | 30° | 30°-35° |
| with the stem | | | |
| Response time in | 50 | 49 | 53-54 |
| days | | | |

I claim:

1. A new and distinct variety of chrysanthemum plant as described and illustrated.
